(19) United States
(12) Patent Application Publication
(10) Pub. No.: US 2009/0028945 A1
(43) Pub. Date: Jan. 29, 2009

(54) Immunoisolative encapsulation system

(75) Inventors: Charles Y. Cheung, Superior, CO (US); Kristi S. Anseth, Boulder, CO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: University of Colorado, Boulder, CO (US)

(21) Appl. No.: 11/447,393

(22) Filed: Jun. 5, 2006

Related U.S. Application Data
(60) Provisional application No. 60/687,590, filed on Jun. 4, 2005.

(51) Int. Cl. A61K 9/00; A61K 39/395; A61P 3/10; A61P 37/06

(52) U.S. Cl. 424/486; 424/158.1

(57) ABSTRACT

A fundamental deficiency with current biological cell encapsulation technology is that passive material barriers cannot protect biological cells from exposure to cytokines and other small, diffusible cytotoxic molecules produced by activated immune cells, subsequently leading to biological cell destruction. The present invention provides an immunoisolative encapsulation system that actively and locally suppresses immune response using Fas receptor binding agents.

### CROSS-REFERENCES TO RELATED APPLICATIONS

This application claims the benefit of U.S. application Ser. No. 60/687,590, filed Jun. 3, 2005, which is herein incorporated by reference in its entirety for all purposes.

### STATEMENT AS TO RIGHTS TO INVENTIONS MADE UNDER FEDERALLY SPONSORED RESEARCH OR DEVELOPMENT

The invention described herein was made with government support under Grant Number EEC0444771 awarded by the National Science Foundation. The United States Government has certain rights in the invention.

### BACKGROUND OF THE INVENTION

Transplantation of biological tissues, such as pancreatic islet transplantation, represents an attractive treatment option for disease states, such as Type I diabetes mellitus. These transplantations, however, require lifelong systemic immunosuppressive therapies to be successful. Encapsulation of biological cells within a cell impermeant polymer matrix provides a passive physical barrier that allows for small molecule diffusion through the barrier but prevents T-cell infiltration and cell contact-mediated destruction of grafted cells. Much of the current research has focused on forming polyionic complexes using sodium alginate mixed with different polycationic compounds to form polymeric barriers for immunoisolation of cells (Orive, G., Hernandez, R. M., Rodriguez Gascon, A., Calafiore, R., Chang, T. M., de Vos, P., Hortelano, G., Hunkeler, D., Lacik, I., and Pedraz, J. L. (2004) History, challenges and perspectives of cell microencapsulation. Trends Biotechnol 22, 87-92).

Microencapsulation systems of biological cells has been accomplished with a polyelectrolyte complex of alginate and polylysine (Lim, F., and Sun, A. M. (1980) Microencapsulated islets as bioartificial endocrine pancreas. Science 210, 908-10). Over the past twenty years, this system has been continually optimized. For example, islets encapsulated in alginate matrices have survived in vivo for a significant duration of time while maintaining euglycemia, up to 6 months (de Vos, P., van Hoogmoed, C. G., van Zanten, J., Netter, S., Strubbe, J. H., and Busscher, H. J. (2003) Long-term biocompatibility, chemistry, and function of microencapsulated pancreatic islets. Biomaterials 24, 305-12). Biocompatibility issues with these types of matrices still persist, however, especially with the choice of polycation used to form these membrane barriers (Strand, B. L., Ryan, T. L., In't Veld, P., Kulseng, B., Rokstad, A. M., Skjak-Brek, G., and Espevik, T. (2001) Poly-L-Lysine induces fibrosis on alginate microcapsules via the induction of cytokines. Cell Transplant 10, 263-75).

Poly(ethylene glycol) (PEG) represents an alternative biomaterial that is biocompatible and has gained FDA approved when incorporated into different drug formulations (http://www.nektar.com/content/pipeline_{—}2). Cruise et al. microencapsulated islets by photopolymerizing PEG diacrylate (PEGDA) to form their passive membrane barriers (Cruise, G. M., Hegre, 0. D., Scharp, D. S., and Hubbell, J. A. (1998) A sensitivity study of the key parameters in the interfacial photopolymerization of poly(ethylene glycol) diacrylate upon porcine islets. Biotechnol Bioeng 57, 655-65). Islets encapsulated in PEDGA were able to maintain normoglycemia in streptozotocin-induced mice for up to 4 months (Cruise, G. M., Hegre, O. D., Lamberti, F. V., Hager, S. R., Hill, R., Scharp, D. S., and Hubbell, J. A. (1999) In vitro and in vivo performance of porcine islets encapsulated in interfacially photopolymerized poly(ethylene glycol) diacrylate membranes. Cell Transplant 8, 293-306). Investigations into the biocompatibility of PEG has shown that in vitro PEG itself does not activate immune cells, such as splenetic lymphocytes and macrophages; however, PEG cannot actively protect islets from cytotoxic molecules produced by pre-activated immune cells (Jang, J. Y., Lee, D. Y., Park, S. J., and Byun, Y. (2004) Immune reactions of lymphocytes and macrophages against PEG-grafted pancreatic islets. Biomaterials 25, 3663-9). This example illustrates one of the fundamental deficiencies with current encapsulation technology, that cytokines and other effector molecules produced by immune cells can still diffuse through these passive barrier matrices to inactivate or destroy transplanted cells (Bottino, R., Lemarchand, P., Trucco, M., and Giannoukakis, N. (2003) Gene- and cell-based therapeutics for type I diabetes mellitus. Gene Ther 10, 875-89; de Vos, P., and Marchetti, P. (2002) Encapsulation of pancreatic islets for transplantation in diabetes: the untouchable islets. Trends Mol Med 8, 363-6; Gray, D. W. (2001) An overview of the immune system with specific reference to membrane encapsulation and islet transplantation. Ann N Y Acad Sci 944, 226-39).

Encapsulation of grafted cells with passive materials may prevent destruction of the cells initiated by cell-cell contact with autoreactive T cells. Such passive barriers, however, cannot protect cells from exposure to cytokines and other effector molecules, such as IL-1, produced by activated immune cells that can subsequently lead to transplanted cell destruction (Bottino, R., Lemarchand, P., Trucco, M., and Giannoukakis, N. (2003) Gene- and cell-based therapeutics for type I diabetes mellitus. Gene Ther 10, 875-8; Mandrup-Poulsen, T., Zumsteg, U., Reimers, J., Pociot, F., Morch, L., Helqvist, S., Dinarello, C. A., and Nerup, J. (1993) Involvement of interleukin 1 and interleukin 1 antagonist in pancreatic beta-cell destruction in insulin-dependent diabetes mellitus. Cytokine 5, 185-91). By providing a novel active immunoisolative encapsulation system comprising Fas receptor binding agents, the present invention fulfills these and other needs in the art.

### BRIEF SUMMARY OF THE INVENTION

It has been discovered that, surprisingly, passive cellular encapsulation systems may be transformed into active barrier systems capable of providing localized immunosuppression by covalently binding Fas receptor binding agents to the polymer matrix of the encapsulation system. Thus, the present invention provides novel immunoisolative encapsulation systems and methods for their use.

In one aspect, the present invention provides an immunoisolative encapsulation system. The system includes a plurality of biological cells encapsulated within a polymer matrix. The polymer matrix is covalently bound to a plurality of Fas receptor binding agents.

In another aspect, the present invention provides a method of inducing apoptosis in a T-cell. The method includes contacting the T-cell with the immunoisolative encapsulation system of the present invention. At least one of the plurality of Fas receptor binding agents is allowed to bind to a Fas receptor on the surface of the T-cell thereby inducing apoptosis.

In another aspect, the present invention provides a method of treating Type I diabetes mellitus in a subject in need of such treatment. The method includes transplanting an immunoisolative encapsulation system into the subject. The immunoisolative encapsulation system includes a plurality of islet cells encapsulated within a polymer matrix covalently bound to a plurality of Fas receptor binding agents. The beta cells (β cells) of the plurality of islet cells are allowed to secrete insulin thereby treating the subject.

In another aspect, the present invention provides a method of locally suppressing an immune response to a transplanted biological material in a subject. The method includes encapsulating the transplanted biological material within a polymer matrix covalently bound to a plurality of Fas receptor binding agents thereby forming an immunoisolative encapsulation system. The immunoisolative encapsulation system is transplanted into the subject. At least one of the plurality of Fas receptor binding agents is allowed to bind to one or more Fas receptors on the surface of a plurality of T-cells, thereby locally suppressing the immune response to the transplanted biological material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A illustrates PEGDA-co-NPA hydrogels photopolymerized prior to conjugation to MAH IgG. FIG. 1B illustrates the reaction of NHS moieties to amine groups of anti-Fas antibodies.

FIG. 2A illustrates indirect ELISA results verifying MAH IgG conjugation to hydrogels resulting from experiments in which MAH IgG solution concentration is increased (0-1 mg/ml) and fraction of NPA is increased (5-50 mol %). FIG. 2B and 2C illustrate the results of sandwich ELISA assays performed on 75/25 and 50/50 PEGDA-co-NPA hydrogels, respectively.

FIG. 3 illustrates the results of cryosectioning and immunostaining of hydrogels not exposed to MAH (3A); and MAH surface-conjugated hydrogels at 0.1 mg/ml (3B) and 0.5 mg/ml (3C).

FIG. 4 illustrates indirect and sandwich ELISAs performed with PEGDA-co-NPA hydrogels prepared at varying ratios (100:0 to 50:50 PEGDA:NPA) conjugated to 0-0.25 mg/ml DX2 anti-Fas MAbs.

FIG. 5 illustrates Annexin V fluorescence experiments comparing the ability of DX2-conjugated hydrogels (5C) to induce apoptosis within Jurkat cells compared to control hydrogels in panels 5A and 5B, which contain no antibody and MAH IgG antibody, respectively.

FIG. 6 illustrates quantitative results from the Annexin V fluorescence experiments shown in FIG. 5.

FIG. 7 illustrates results comparing apoptosis induction between 19.2 Fas-insensitive T cells and Fas-sensitive Jurkat cells exposed to DX2-conjugated hydrogels and hydrogels without DX2 antibodies.

FIG. 8 illustrates a brightfield image of islets encapsulated within double-layered PEGDA hydrogel taken with 5× objective magnification, wherein the arrows represent boundaries of both inner and outer hydrogels.

FIG. 9 represents fluorescent images of islets encapsulated in both unconjugated (9A, 9C) and DX2-conjugated (9B, 9D) hydrogels taken at 10× objective magnification, wherein green staining represents live cells, and red staining represents dead cells.

FIG. 10 represents data from an indirect ELISA verifying the presence of DX2 on the surface of DX2-conjugated islet encapsulated hydrogels, whereas no antibody is evident within unconjugated hydrogels.

### DETAILED DESCRIPTION OF THE INVENTION

### Definitions

“Peptide” refers to a polymer in which the monomers are amino acids and are joined together through amide bonds, alternatively referred to as a polypeptide. A protein is a peptide or polypeptide or two or more peptides joined together through non-amide bonds (e.g. disulfide bridges, hydrogen bonds, and or hydrophobic interactions). When the amino acids are α-amino acids, either the L-optical isomer or the D-optical isomer can be used. Additionally, unnatural amino acids, for example, β-alanine, phenylglycine and homoarginine are also included. Commonly encountered amino acids that are not gene-encoded may also be used in the present invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

As used herein, “amino acid” refers to a group of water-soluble compounds that possess both a carboxyl and an amino group attached to the same carbon atom. Amino acids can be represented by the general formula NH₂—CHR—COOH where R may be hydrogen or an organic group, which may be nonpolar, basic, acidic, or polar. As used herein, “amino acid” refers to both the amino acid radical and the non-radical free amino acid.

### Immunoisolative Encapsulation System

In one aspect, the present invention provides an immunoisolative encapsulation system. The system includes a plurality of biological cells encapsulated within a polymer matrix. The polymer matrix is covalently bound to a plurality of Fas receptor binding agents. At least some of the Fas receptor binding agents are covalently bound to a region of the polymer matrix suitable for binding to Fas receptors present on the surface of lymphocytes. In some embodiments, the Fas receptor binding agents are covalently bound to the surface (i.e. the outer portion of the polymer matrix in contact with exterior fluids when present) of the polymer matrix.

A variety of encapsulation technologies are known in the art and are useful in the present invention. See, for example, U.S. Pat. Nos. 6,911,227, 6,399,341, 6,352,707, 6,303,136, 6,126, 936, 5,916,790, each of which describes various encapsulation technologies that are herein incorporated by reference in their entirety. Preferred polymer matrices for use in the present invention are cell impermeable and biocompatible when transplanted (e.g. grafted) into a host organism (e.g. a mammal such as a human). The polymer matrix contains pores sufficiently small to prevent the movement of biological cells into or out of the polymer matrix. Where the biological cells are secretory cells, the polymer matrix contains pores sufficiently large to allow the movement of biological molecules (e.g. insulin) out of the polymer matrix.

In some embodiments, the polymer matrix is a hydrogel. As used herein, the term “hydrogel” refers to any naturally-occurring or synthetic hydrophilic material capable of retaining liquid within its structure, but does not dissolve in the liquid. Typically, hydrogels swell in aqueous solution to an equilibrium volume and maintain their shape. See Kroschwitz et al., Concise Encyclopedia of Polymer Science and Engineering, New York: Wiley. xxix, 1341 (1990); Mark et al., Encyclopedia of polymer Science and Engineering, 2nd ed. New York: Wiley (1985). Hydrogels are typically comprised of hydrophilic monomers, cross-linkers, and initiators. Hydrophilic monomers bear hydrophilic chemical moieties such as NH₂, OH, COOH, and CONH₂. Crosslinking may be achieved through covalent bonding, ionic bonding, hydrogen bonding, hydrophobic interactions, and dipole-dipole interactions. Hydrogels based on the following hydrophilic monomers are useful: 2-hydroxyethyl methacrylates (HEMA), ethylene glycol dimethacrylates (EGDMA), polyethylene glycol dimethacrylates, polyethylene glycol diacrylate (PEGDA), trimethacrylates (e.g., trimethylolpropane trimethacrylate (TMPTMA), ethyleneglycol dimethacrylate (EGDMA), triethyleneglycol dimethacrylate (TEGDMA), bis(2-methacryloxyethyl)ester of isophthalic acid (MEI), bis(2-methacryloxyethyl)ester of terephthalic acid (MET), bis(2-methacryloxyethyl)ester of phthalic acid (MEP), 2,2-bis-(4-methacryloxy phenyl)propane(BisMA), 2,2-bis[4-(2-methacrylyloxyethoxy)phenyl]propane (BisEMA), 2,2,-bis[4-(3-methacrylyloxypropoxy)phenyl]propane (BisPMA), hexafluoro-1,5-pentanediol dimethacrylate (HFPDMA), bis-(2-methacrylyloxyethoxyhexafluoro-2-propyl)benzene [Bis(MEHFP).phi.], 1,6-bis(methacrylyloxy-2-ethoxycarbonylamino)-2,4,4-trimethylhexan (UEDMA), spiro orthocarbonates, and the derivatives thereof.

In some embodiments, the hydrogel is a double layered hydrogel comprising an inner hydrogel and an outer hydrogel, wherein the Fas receptor binding agent is covalently bound to the outer hydrogel. The inner layer hydrogel and outer layer hydrogel are typically composed of different polymer materials. The biological cells may be encapsulated within the inner hydrogel. In some embodiments, the double layered hydrogel prevents the Fas receptor binding agent covalently bound to the outer layer from interacting with the biological cells within the inner layer. This may be especially important where the biological cell expresses Fas receptor.

In some embodiments, the polymer matrix is constructed in the presence of biological cells. In these embodiments, the polymer matrix is capable of being constructed under conditions that are non-toxic, or at least substantially non-toxic, to the biological cells being encapsulated. In some embodiments, the polymer matrix is crosslinked using photopolymerization techniques known in the art. Where photopolymerization is employed, one skilled in the art will immediately understand that a photoinitiator is employed, such as 1-[4-(2-hydroxyethoxy)-phenyl]-2-hydroxy-2-methyl-1-propane-1-one, ethyl eosin, eosin Y, fluorescein, 2,2-dimethoxy, 2-phenylacetophenone, 2-methyl, 2-phenylacetonphenone, I2959, camphorquinone, rose bengal, methylene blue, erythosin, phloxime, thionine, riboflavin, and methyl green.

In certain embodiments, the polymer matrix is composed of monomers or polymerized monomers (sometimes referred to as macromers) selected from polyethylene glycol (PEG) (e.g. polyethylene glycol dimethacrylate (PEGDA)), poly(ethylene oxide) (PEO), poly(vinyl alcohol) (PVA), poly(vinylpyrrolidone) (PVP), poly(ethyloxazoline) (PEOX), poly(amino acids), and polysaccharides. A variety of crosslinkers may be employed where a polyethylene glycol based polymer matrix is desired, such as N-hydroxysuccinimide-PEG-acrylate. PEG represents a functionally versatile inert polymer material that is easily modified to attach Fas receptor binding agents to actively promote and assist the long-term survival of encapsulated biological cells by downregulating the localized immune response. Investigators have previously incorporated polymerizable antibodies and antibody fragments into hydrogels by free radical polymerization, but with the sole purpose for the diagnostic detection of antigens and biosensor applications ( Lu, Z., Kopeckova, P., and Kopecek, J. (2003) Antigen responsive hydrogels based on polymerizable antibody Fab′ fragment. MACROMOLECULAR BIOSCIENCE 3, 296-300, Miyata, T., Asami, N., and Uragami, T. (1999) A reversibly antigen-responsive hydrogel. Nature 399, 766-9). Thus, in some embodiments, the polymer matrix of the present invention is a hydrogel comprising polyethylene glycol polymers. The hydrogel may be a PEGDA-co-NPA hydrogel.

The polymer matrix may encapsulate biological cells through microencapsulation of small groups of cells (e.g. pancreatic islets) or macroencapsulation of larger groups of cells, such as a plurality of pancreatic islets, portions of organs or tissues, or complete organs.

In the encapsulation systems of the present invention, the polymer matrix is covalently bound to a Fas receptor binding agent. A Fas receptor binding agent, as used herein, refers to a molecule that specifically binds to the Fas receptor and is capable of inducing apoptosis in Fas expression lymphocytes (see, for example, U.S. Pat. No. 6,235,878). A variety of Fas receptor binding agents are useful in the present invention. Fas is a 45 kD cell surface protein belonging to the TNF receptor family of proteins that can induce apoptosis when engaged with Fas Ligand (FasL). In some embodiments, the Fas receptor binding agent is Fas Ligand protein or a functional fragment thereof. Fas-mediated. apoptosis is normally involved in clonal deletion of autoreactive T cells by negative selection (Palmer, E. (2003) Negative selection--clearing out the bad apples from the T-cell repertoire. Nat Rev Immunol 3, 383-91). The Fas pathway is also responsible for the elimination of activated T cells following the completion of the immune response to infection. Many tissues and cell lines weakly express Fas, but Fas is highly expressed in activated, mature lymphocytes such as T-cells (alternatively referred to as t-lymphocytes), making lymphocytes highly susceptible to Fas-mediated apoptosis (Nagata, S., and Golstein, P. (1995) The Fas death factor. Science 267, 1449-56, Timmer, T., de Vries, E. G., and de Jong, S. (2002) Fas receptor-mediated apoptosis: a clinical application? J Pathol 196, 125-34). FasL is the natural homotrimeric protein binding ligand for Fas, binding to three Fas receptor molecules to induce apoptosis (Timmer, T., de Vries, E. G., and de Jong, S. (2002) Fas receptor-mediated apoptosis: a clinical application? J Pathol 196, 125-34). FasL expression is induced upon activation of cytotoxic T lymphocytes, which can then destroy Fas-expressing activated lymphocytes (Nagata, S., and Golstein, P. (1995) The Fas death factor. Science 267, 1449-56). Fas activation requires FasL multimerization or cross-linking of Fas receptors to initiate the signal transduction cascade for apoptosis (Holler, N., Tardivel, A., Kovacsovics-Bankowski, M., Hertig, S., Gaide, O., Martinon, F., Tinel, A., Deperthes, D., Calderara, S., Schulthess, T., Engel, J., Schneider, P., and Tschopp, J. (2003) Two adjacent trimeric Fas ligands are required for Fas signaling and formation of a death-inducing signaling complex. Mol Cell Biol 23, 1428-40). Binding of MAbs to Fas receptors can also induce apoptosis. The multimeric nature of Anti-Fas IgM antibodies effectively induce apoptosis by allowing for multiple cooperative binding events to cross-link several Fas receptors simultaneously on cell surfaces (Fadeel, B., Thorpe, C. J., Yonehara, S., and Chiodi, F. (1997) Anti-Fas IgG1 antibodies recognizing the same epitope of Fas/APO-1 mediate different biological effects in vitro. Int Immunol 9, 201-9, Komada, Y., Inaba, H., Li, Q. S., Azuma, E., Zhou, Y. W., Yamamoto, H., and Sakurai, M. (1999) Epitopes and functional responses defined by a panel of anti-Fas (CD95) monoclonal antibodies. Hybridoma 18, 391-8). IgM subclass anti-Fas antibodies induce apoptosis effectively due to their ability to simultaneously crosslinking multiple Fas receptors in the absence of exogenously added crosslinking molecules (Dhein, J., Daniel, P. T., Trauth, B. C., Oehm, A., Moller, P., and Krammer, P. H. (1992) Induction of apoptosis by monoclonal antibody anti-APO-1 class switch variants is dependent on cross-linking of APO-1 cell surface antigens. J Immunol 149, 3166-73). As demonstrated herein, IgG subclass anti-Fas antibodies effectively induce apoptosis when concentrated on a polymer matrix. DX2 is an anti-Fas monoclonal antibody (IgG1 subclone) that can induce apoptosis in human T cells lines in solution upon antibody crosslinking (Komada, Y., Inaba, H., Li, Q. S., Azuma, E., Zhou, Y. W., Yamamoto, H., and Sakurai, M. (1999) Epitopes and functional responses defined by a panel of anti-Fas (CD95) monoclonal antibodies. Hybridoma 18, 391-8, Cifone, M. G., De Maria, R., Roncaioli, P., Rippo, M. R., Azuma, M., Lanier, L. L., Santoni, A., and Testi, R. (1994) Apoptotic signaling through CD95 (Fas/Apo-1) activates an acidic sphingomyelinase. J Exp Med 180, 1547-52).

Thus, in some embodiments, the Fas receptor binding agent employed in the present invention is an anti-Fas antibody, such as a monoclonal anti-Fas antibody. As used herein, the term “antibody” refers to a protein functionally defined as a binding protein and structurally defined as comprising an amino acid sequence that is recognized by one of skill as being derived from the framework region of an immunoglobulin encoding gene of animal producing antibodies. An antibody can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. In some embodiments, the Fas receptor binding agent is an IgG or IgM anti-Fas antibody.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Useful antibodies include single chain antibodies (antibodies that exist as a single polypeptide chain), and single chain Fv antibodies (sFv or scFv) in which a variable heavy and a variable light chain are joined together (directly or through a peptide linker) to form a continuous polypeptide. The single chain Fv antibody is a covalently linked VH-VL heterodimer which may be expressed from a nucleic acid including VH- and VL- encoding sequences either joined directly or joined by a peptide-encoding linker. Huston, et al. (1988) Proc. Nat. Acad. Sci. USA, 85: 5879-5883. While the VH and VL are connected to each as a single polypeptide chain, the VH and VL domains associate non-covalently. The two chains can be encoded on the same or on different replicons; the important point is that the two antibody chains in each Fab molecule assemble post-translationally and the dimer is incorporated into the phage particle via linkage of one of the chains to g3p (see, e.g., U.S. Pat. No. 5,733,743). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778). Thus, useful antibodies include all whole antibodies, or antibody fragments such as scFv, Fv, Fab and disulfide linked Fv (Reiter et al. (1995) Protein Eng. 8: 1323-1331). Antibodies can also include diantibodies and miniantibodies.

The Fas receptor binding agent may be covalently attached to the polymer matrix using a polymer matrix reactive functional group. Where the Fas receptor binding agent is a peptide or protein, the polymer matrix reactive functional group is reactive with a protein or peptide reactive group, such as an amino moiety or carboxylic acid moiety. Bioconjugation techniques useful for conjugation of proteins and other biomolecules to polymers are generally well known in the art, and are reviewed in more detail, for example in March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

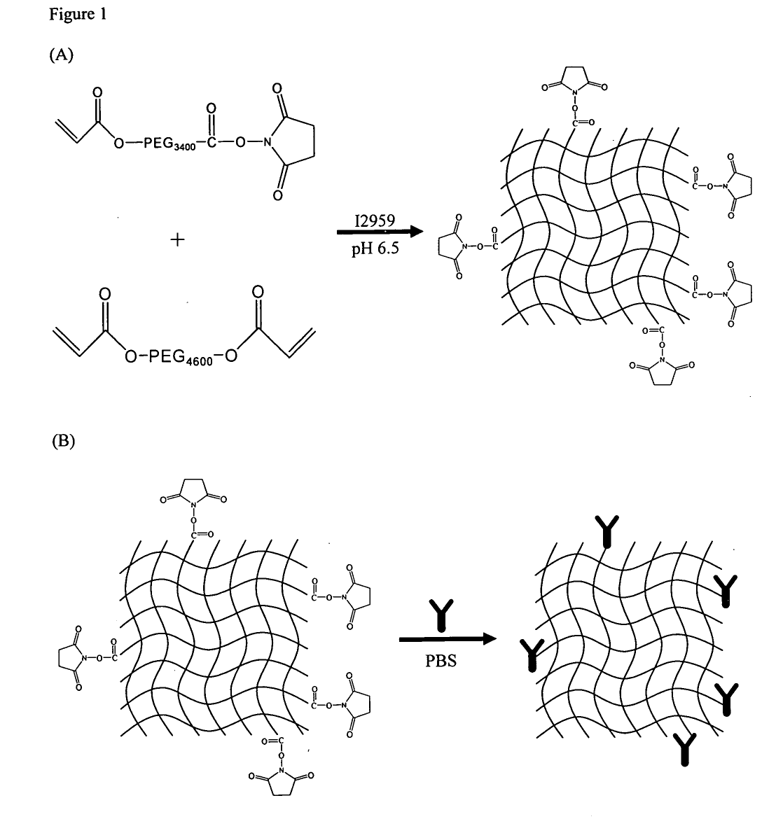

In some embodiments, the polymer matrix reactive functional group is present at the terminal portion of the polymers thereby providing polymer matrix reactive functional groups at the surfaces of the polymer matrix (FIG. 1). The surface polymer matrix reactive functional group facilitates covalent attachment of the Fas receptor binding agent at the surface of the polymer matrix. Thus, in some embodiments the plurality of Fas receptor binding agents are covalently bound to the surface of the polymer matrix.

Functional groups and classes of reactions useful in practicing the present invention are generally those that are well known in the art of bioconjugate chemistry. Currently favored classes of reactions available with reactive known reactive groups are those which proceed under relatively mild conditions amenable to biological cell survival. These include, but are not limited to, nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition).

Useful reactive functional groups include, for example:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc.

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups, which can be converted to disulfides or reacted with acyl halides;

(h) amine or sulfhydryl groups, which can be, for example, acylated, alkylated or oxidized;

(i) alkenes, which can undergo, for example, cycloadditions, acylation, Michael addition, etc;

(j) epoxides, which can react with, for example, amines and hydroxyl compounds; and

(k) phosphoramidites and other standard functional groups useful in nucleic acid synthesis.

Linkers may also be employed to attach the Fas receptor binding agent to the polymer matrix. Linkers may include reactive groups at the point of attachment to the Fas receptor binding agent and/or the polymer matrix. Any appropriate linker may be used in the present invention, including substituted or unsubstituted alkylene, substituted or unsubstituted heteroalkylene, substituted or unsubstituted cycoalkylene, substituted or unsubstituted heterocycloalkylene, substituted or unsubstituted arylene, and substituted or unsubstituted heteroarylene. Other useful linkers include those having a polyester backbone (e.g. polyethylene glycol), nucleic acid backbones, amino acid backbones, and derivatives thereof. A wide variety of useful linkers are commercially available (e.g. polyethylene glycol based linkers such as those available from Nektar, Inc. of Huntsville, Ala.).

A variety of biological cells may be encapsulated within a polymer matrix of the present invention. In some embodiments, the biological cells are secretory cells (i.e. biological cells capable of producing and secreting biological compounds useful in treating a disease state). The biological cells may be selected from hepatocytes, parathyroid cells, pancreatic cells, or transformed cell lines (e.g. Chinese hamster ovary (CHO) cells, myoblast cell lines). In some embodiments, the transformed cell lines produce and secrete therapeutic recombinant proteins or peptides. In some embodiments, the pancreatic cells are islet cells. The islet cells may comprise, or may be, insulin producing β-cells. Other examples of cells that can be encapsulated are human foreskin fibroblasts, beta cell insulomas, lymphoblastic leukemia cells, mouse 3T3 fibroblasts, dopamine secreting ventral mesencephalon cells, neuroblastoid cells, and adrenal medulla cells. As can be seen from this partial list, cells of all types, including dermal, neural, blood, organ, muscle, glandular, reproductive, and immune system cells can be encapsulated successfully.

The thickness of the polymer matrix may be adjusted using a variety of techniques well known in the art, such as the duration of the irradiation during photopolymerization. One skilled in the art will immediately recognize that the size and thickness of the polymer matrix depends on a variety of factors, such as the number of biological cells to be encapsulated, and the desired rigidity of the polymer matrix. The polymer matrix may be of any appropriate thickness. In some embodiments where macroencapsulation is employed, the thickness varies from 0.1 mm to 100 mm, 1 mm to 50 mm, or from 3 mm to 10 mm. Where microencapsulation is employed, one skilled in the art will recognize that the thickness of the polymer matrix will depend on the number and type of cell being encapsulated. For example, where a single cell is encapsulated, the thickness may range from 5 to 100 μM, from 10 to 50 μM, or from 20 to 30 μM. Where islets are encapsulated, the thickness may range from 100 to 800 μM, or form 200 to 500 μM.

The shape of the polymer matrix formed can be controlled by shaping the reaction mix prior to polymerization. Spheres can be formed by emulsion with a non-miscible liquid such as oil, coextrusion with such a liquid, or coextrusion with air. Semi-spherical shapes may be obtained using a micro-needle injection device. Cylinders may be formed by casting or extrusion, and slabs and discoidal shapes can be formed by casting. Additionally, the shape may be formed in relationship to an internal supporting structure such as a screening network of stable polymers (e.g. an alginate gel or a woven polymer fiber) or nontoxic metals.

### Methods

In another aspect, the present invention provides a method of inducing apoptosis in a T-cell. The method includes contacting the T-cell with the immunoisolative encapsulation system of the present invention (which is described above and equally applicable to the methods of the present invention). The system includes a plurality of biological cells encapsulated within a polymer matrix. The polymer matrix is covalently bound to a plurality of Fas receptor binding agents. At least one (or a sufficient number) of the plurality of Fas receptor binding agents is allowed to bind to a Fas receptor on the surface of the T-cell thereby inducing apoptosis. In some embodiments, the T cell is an activated, mature T-cell that highly expresses the Fas receptor (Nagata, S., and Golstein, P. (1995) The Fas death factor. Science 267, 1449-56, Timmer, T., de Vries, E. G., and de Jong, S. (2002) Fas receptor-mediated apoptosis: a clinical application? J Pathol 196, 125-34). Engagement of the cell surface T cell receptor (TCR) initiates T cell activation. Signal transduction from the TCR leads to T cell activation and changes in T cell gene expression, physiology, and function, including the secretion of cytokines and expression of the Fas receptor. In some embodiments, the contacting is performed in vivo, such as in a mammal (e.g. a human).

In another aspect, the present invention provides a method of treating Type I diabetes mellitus in a subject in need of such treatment. The method includes transplanting an immunoisolative encapsulation system into the subject. The immunoisolative encapsulation system includes a plurality of islet cells encapsulated within a polymer matrix covalently bound to a plurality of Fas receptor binding agents. The beta cells (β cells) of the plurality of islet cells are allowed to secrete insulin thereby treating the subject. Immunoisolative encapsulation systems with islet cells within the polymer matrix covalently bound to Fas receptor binding reagents are described above and equally applicable to the present methods. Typically, the immunoisolative encapsulation system is grafted onto the pancreas of the subject using techniques known in the art.

As described above, transplantation of a biological material (e.g. biological cells, tissue, organs, etc.) into a host organism (e.g. a mammal such as a human) is typically hindered by the host immune response resulting in destruction of the biological material. The immunoisolative encapsulation system of the present invention, however, is capable of inducing apoptosis in mature lymphocytes expressing high levels of Fas receptor. The system provides Fas receptor binding agents covalently bound to the polymer matrix of the immunoisolative encapsulation system that bind to the Fas receptor lymphocytes thereby inducing apoptosis. The immunoisolative encapsulation system of the present invention, therefore, locally suppresses the immune response by inducing apoptosis in lymphocytes within the immediate vicinity of the polymer matrix. Thus, in another aspect, the present invention provides a method of locally suppressing an immune response to a transplanted biological material in a subject. The method includes encapsulating the transplanted biological material within a polymer matrix covalently bound to a plurality of Fas receptor binding agents thereby forming an immunoisolative encapsulation system. The immunoisolative encapsulation system is transplanted into the subject. At least one (or a sufficient number) of the plurality of Fas receptor binding agents is allowed to bind to one or more Fas receptors on the surface of a plurality of T-cells, thereby locally suppressing the immune response to the transplanted biological material.

In some embodiments, the subject is a mammal, such as a human. The biological material comprises biological cells and includes, for example, transplantable organs or portions thereof. In some embodiments, the biological material are biological cells, as described above in the discussion of the immunoisolative encapsulation system of the present invention. Thus, ins some embodiments, the transplanted biological material is transplanted hepatocytes, parathyroid cells , or pancreatic cells (e.g. islet cells).

### EXAMPLES

The following examples are offered to illustrate, but not to limit the claimed invention.

### Preparation and Characterization of Mouse Anti-Human IgG Conjugated Hydrogels

Polyethylene diacrylate (PEGDA, MW=4600 g/mol) was polymerized with N-hydroxysuccinimide-PEG-acrylate (NPA, MW=3400 g/mol) by UV-initiated photopolymerization to form PEGDA-co-NPA hydrogels with PEGDA-to-NPA ratios ranging from 95/5 to 50/50 (wt) using a 0.05% (wt) UV photoinitiator, 1-[4-(2-hydroxyethoxy)-phenyl]-2-hydroxy-2-methyl-1-propane-1-one (I2959, Ciba Geigy, Newport, Del.). Hydrogels constituents were dissolved in 0.1 SM sodium phosphate buffer, pH 6.5, and hydrogels were prepared by photopolymerization for 15 minutes at 365 nm. Mouse anti-human IgG (MAH IgG, Fitzgerald Industries International, Concord, Mass.) was then conjugated to these hydrogels at concentrations ranging from 0.05-1 mg/ml for 1 hr in PBS. Hydrogels were then incubated with 50 mM glycine in PBS to quench the reactivity of any unreacted NHS groups remaining in the gel.

The level of MAH IgG conjugated to the surface of the hydrogels was assessed by a modified indirect ELISA using goat anti-mouse horseradish peroxidase (GAM HRP) (Jackson Immunoresearch, West Grove, Pa.) to detect surface-conjugated MAH IgG. A modified sandwich ELISA was prepared to determine the extent to which MAH IgG retained the ability to recognize its antigen, human IgG, after conjugation. 75/25 or 50/50 PEGDA-co-NPA hydrogels conjugated with MAH IgG were exposed to human IgG at concentrations ranging from 0-50 ng/ml, followed by incubation with goat anti-human HRP. Hydrogels in both assays were incubated with Ultra-TMB (Pierce Biotechnology, Rockford, Ill.), and quantitative absorbance measurements were taken at 450 nm.

### Immunostainins and Cryosectioning of Hydrogels

75/25 PEGDA-co-NPA hydrogels were polymerized and conjugated with 0-0.5 mg/ml MAH IgG, as described in the previous section. Hydrogels were then soaked in Cryo-Gel (Instrumedics, Hackensack, N.J.), snap-frozen in liquid N₂ and sectioned into 50 μm sections with a Leica CM 1850 cryostat (Leica, Bannockburn, Ill.) and placed onto glass slides. The sections were then soaked three times in PBS for 5 minutes and blocked with 1% BSA in PBS for 1 hour. After an additional PBS wash, the sections were then immunostained with Alexa 488-conjugated goat anti-mouse F(ab′)² antibody fragments for 1 hour. The sections were washed three times with PBS, then mounted with Biomeda Gel/Mount (Biomeda, Foster City, Calif.) supplemented with 1,4-diazabicyclo-[2,2,2]octane and coverslipped. Fluorescence images were taken with a Zeiss Axioplan 2 confocal microscope (Zeiss, Thornwood, N.Y.).

### Preparation and Characterization of Anti-Fas MAb Conjugated Hydrogels

PEGDA-co-NPA hydrogels were prepared at 100/0 to 50/50 PEGDA/NPA ratios as described in the previous section. These hydrogels were then conjugated with 0-0.25 mg/ml of anti-Fas MAbs (DX2 clone, R&D Systems, Minneapolis, Minn.) using the same reaction conditions as described in the previously for MAH IgG. The reactivity of any unreacted NHS groups within the gels was then quenching upon addition of 50 mM glycine methyl ester (Sigma-Aldrich, St. Louis, Mo.). Both modified indirect and sandwich ELISAs were performed as described previously. The degree of DX2 conjugation was assessed by indirect ELISAs using goat anti-mouse HRP. Sandwich ELISAs to verify DX2 recognition and binding of recombinant human Fas (Peprotech, Rocky Hill, N.J.) was performed utilizing goat anti-Fas PAb (R&D Systems) for the sandwich antibody and donkey anti-goat HRP for secondary detection (Jackson Immunoresearch).

### Apoptosis Assessment by Annexin V Assay

Jurkat cells were cultured with RPMI 1640 supplemented with 100 u/ml Penicillin/Streptomycin and Fungizone and 10% fetal bovine serum (FBS). For apoptosis induction experiments, 75/25 PEGDA/NPA hydrogels were polymerized in 96-well TCPS under aseptic conditions. These gels were soaked in sterile-PBS for 5 minutes, then conjugated to 0.25 mg/ml MAH IgG, or 0.25 mg/ml DX2 anti-Fas MAbs in PBS. After washing and blocking remaining reactive sites on hydrogels using 50 mM glycine methyl ether, 5,000 or 10,000 Jurkat cells were added to the gels and the cells were allowed to incubate suspended atop these hydrogels for 2 days at 37° C. Jurkat cells were then removed and placed into fresh wells following the incubation period. The Annexin Apoptosis Assay (Invitrogen, Carlsbad, Calif.) was then used to determine the extent of apoptosis. Cells were stained with annexin V-FITC conjugate, then counterstained with propidium iodide (PI), which allows for the differentiation between necrotic and apoptotic cells. The cells were resuspended in RPMI without phenol red and supplemented with 10% FBS, and images were taken with a Nikon Eclipse TE300 fluorescent microscope (Nikon, Lewisville, Tex.). The fraction of apoptotic and necrotic cells within each condition tested was quantitated by counting cells within 4 random fields of view. Approximately 150-400 cells were counted per field of view.

Additional apoptosis studies compared the ability of DX2-conjugated hydrogels to induce apoptosis within Fas-sensitive (Jurkat) and Fas-insensitive (I9.2) T cell lines. 75/25 PEGDA/NPA hydrogels were conjugated with either no DX2 or 0.25 mg/ml DX2 in the same manner as stated above. 10,000 cells/well of either Jurkat cells or I9.2 cells (ATCC, Manassas, Va.), a caspase-8mutant Jurkat cell line that is Fas-insensitive, were then added to these hydrogels. Annexin V-FITC binding studies were then performed following two days incubation. Quantitation of the fraction of apoptotic cells with all samples was calculated using the same techniques as described above.

### Encapsulation of Balb/c Islets in PEG Hydrogels and Incubation with Jurkat Cells

Approximately 20 freshly isolated islets from Balb/c mice pancreases were encapsulated into 3/32″ diameter 10% (wt) PEG diacrylate (PEGDA, MW=4600 g/mol) hydrogels prepared within teflon molds by UV-initiated photopolymerization using a 0.05% (wt) I2959 as a UV photoinitiator, at 365 nm for 10 minutes. These islet-containing hydrogels were then placed into ⅛″ diameter Teflon molds and a second layer was polymerized consisting of a 75%/25% mixture of PEGDA4600NHS-PEG-Acrylate (NPA, MW=5000) with 0.05% I2959 for an additional 10 minutes at 365 nm. These islet-containing double-layered hydrogels were then conjugated to 0.25 mg/ml anti-Fas monoclonal antibodies (MAbs) (IgG1 subclass, DX2 subclone) for 1 hour at 37° C. Control non-conjugated hydrogels were exposed to PBS during this conjugation step. All hydrogels were washed with 1 ml of PBS following conjugation, then placed into 300 μl RPMI 1640 supplemented with 10% FBS in a 96-well tissue culture plate for 2 hours in a 37° C. incubator. After this 2 hour period, the media was removed from the hydrogels and media containing 10,000 Jurkat T cell clones was added per gel. The hydrogels were then placed in a 37° C. incubator exposed to Jurkat cells for 2 days.

### LIVE/DEAD Assessment of Encapsulated Islets

Islet-encapsulated hydrogels were placed into 300 μl media immediately after removal of Jurkat cells and allowed to recover at 37° C. overnight prior to assaying for islet cell viability. 200 μl of LIVE/DEAD® Viability/Cytotoxicity stain (Invitrogen, Carlsbad, Calif.) was added to each hydrogel containing encapsulated islets. Hydrogels were incubated at 37° C. for 30 minutes, then washed in PBS. Islet viability within the hydrogels was assessed using a Zeiss Axioplan 2 confocal microscope (Zeiss, Thornwood, N.Y.). Fluorescent images of islets were taken at 10× objective magnification.

### Insulin Secretion of Encapsulated Islets upon Glucose Stimulation

Islet-encapsulated hydrogels were placed into low glucose solution (1.1 mM glucose in Krebs buffer, 115 mM NaCl, 5 mM KCl, 25 mM NaHCO₃, 1 mM MgCl₂, 1 mM CaCl₂, 2.5 mM HEPES) to synchronize beta cells to baseline insulin production levels for 1 hour at 37° C. The hydrogels were then placed in high glucose solution (16.7 mM glucose in Krebs buffer) to determine the response of the encapsulated islets to glucose stimulation for 1 hr at 37° C. The high glucose solution was then collected and the level of insulin produced by the islets in response to glucose stimulation was measured using a mouse insulin ELISA kit (Mercodia Inc., Winston Salem, N.C.). ATP content within the encapsulated cells was measured using the CellTiter-Glo Luminescent Cell Viability Assay (Promega, Madison, Wis.). Insulin production levels were normalized to ATP content to account for the variability in cell number between the hydrogels.

### ELISA for Surface Conjugated Anti-Fas MAb on Islet-Encapsulated Hydrogels

The level of surface-conjugated DX2 anti-Fas MAb on the islet-encapsulated hydrogels was quantitated using a modified indirect ELISA with goat anti-mouse horseradish peroxidase (GAM HRP) (Jackson Immunoresearch, West Grove, Pa.). Hydrogels were placed in 96-well polystyrene plates blocked with 5% BSA in PBS. The hydrogels were washed four times with PBS/0.1% Tween (PBS-T), then incubated for 1 hour with GAM HRP. The hydrogels were then washed 4 times with PBS-T, and the level of HRP present on the surface of the hydrogels was assessed with the Ultra-TMB substrate (Pierce Biotechnology, Rockford, Ill.). 2M sulfuric acid was added to the TMB solution to quench the reaction, and quantitative absorbance measurements were taken at 450 nm.

### Apoptosis Assessment of Islet-Encapsulated Hydrogels by Annexin V Apoptosis Assay

Apoptosis of Jurkat cells using islet-encapsulated hydrogels was determined using the Annexin V Apoptosis Assay as described above.

### Results

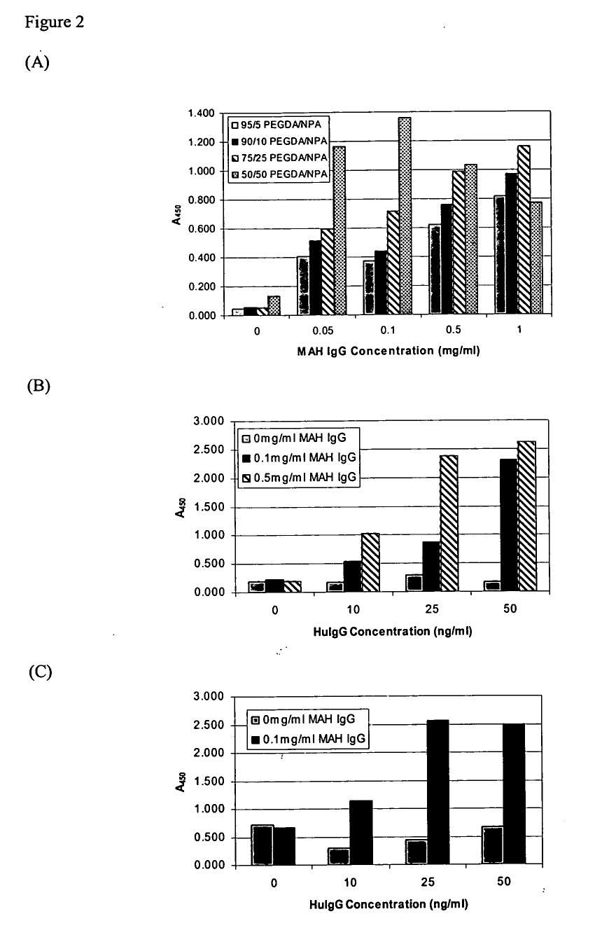

Preliminary experiments were performed with hydrogels prepared in the absence of pancreatic islets to assess the feasibility and functionality of antibody conjugation techniques onto the surfaces of our hydrogels. PEGDA-co-NPA hydrogels were photopolymerized prior to conjugation to MAH IgG (FIG. 1A). NHS moieties were utilized to react to amine groups in the antibodies (FIG. 1B). Preliminary results showed that a high level of MAH IgG was conjugated to the surface of pre-polymerized PEGDA-co-NPA hydrogels. Indirect ELISA assays showed higher levels of MAH IgG conjugated to these hydrogels upon both increasing MAH IgG solution concentration (0-1 mg/ml) and fraction of NPA (5-50 mol %) comprising the hydrogels (FIG. 2A). The highest degree of conjugation was observed with 50/50 PEGDA-co-NPA gels, however, these gels were less mechanically robust due to the lower quantity of crosslinking PEGDA monomer present within these gels. In addition, minimal color formation was evident when 100/0 PEGDA/NPA control gels were reacted with MAH IgG antibodies, indicating that the presence of antibody was due to conjugation to NPA groups and not due to physical adsorption of the antibodies to the hydrogel matrix (data not shown). Sandwich ELISA assays performed on 75/25 and 50/50 PEGDA-co-NPA hydrogels showed that conjugated MAH IgG retained the ability to recognize human IgG antigen, with greater detection sensitivity with gels that contain higher quantities of conjugated MAH IgG (FIG. 2B, 75/25 gels). High nonspecific background absorbance was observed with 50/50 gels in the absence of human IgG (FIG. 2C, 50/50 gels). Detection levels for human IgG become saturated at 25 ng/ml with both gel compositions. These ELISA results indicate that monoclonal antibodies can be conjugated to a high degree to pre-formed PEGDA-co-NPA hydrogels without significantly compromising the abilities of the antibodies to recognize their antigens.

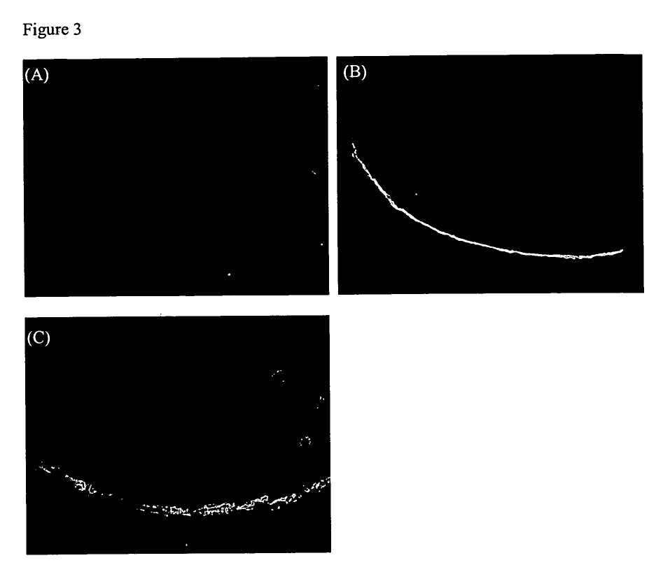

Cryosectioning and immunostaining of MAH-conjugated hydrogels showed that MAH IgG conjugation was limited to the hydrogel surface, as indicated by the lack of fluorescence observed within the interior of the gels (FIG. 3). No fluorescence was observed with hydrogels not exposed to MAH (FIG. 3A). In addition, a thicker band of fluorescence on the hydrogel surfaces was evident with higher concentrations of MAH used for conjugation, which may be indicative of greater degrees of surface conjugation (FIG. 3B&C).

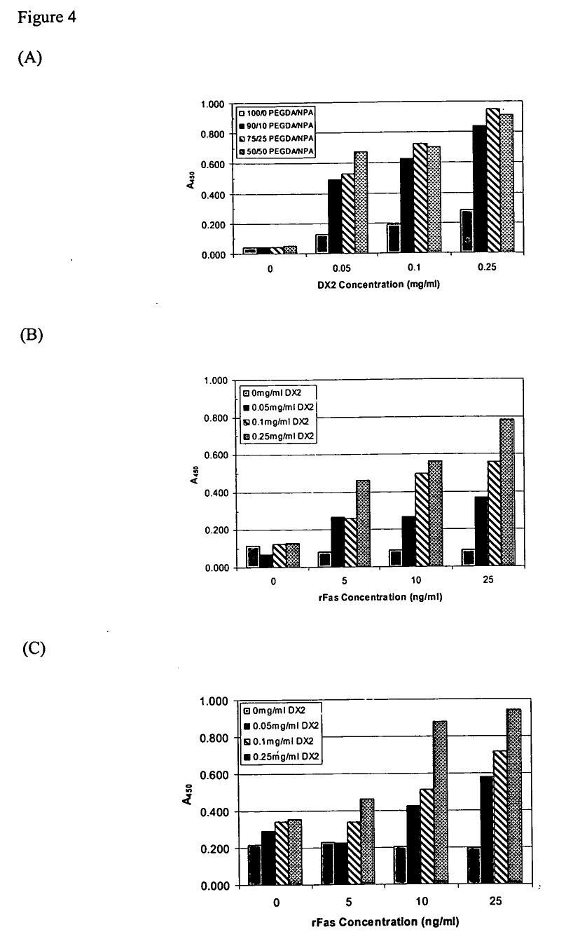

Conjugation of DX2 anti-Fas MAbs to PEGDA-co-NPA hydrogels was performed using the methodology and conditions developed for MAH IgG discussed in the above section. Indirect and sandwich ELISAs were performed with PEGDA-co-NPA hydrogels prepared at varying ratios (100:0 to 50:50 PEGDA:NPA). These assays show similar trends for detection of protein conjugation and antigen recognition by DX2, respectively, as seen with ELISAs with model MAH IgG antibodies (FIG. 4). A higher degree of DX2 conjugation was observed with higher concentrations of antibody added for conjugation (FIG. 4A). However, minimal differences in conjugation efficiency were observed with gels polymerized with different concentrations of NPA, which is in contrast to MAH IgG results. Greater sensitivity of rFas detection was evident with higher concentrations of DX2 conjugated to both 75/25 and 50/50 PEGDA/NPA hydrogels, however significant background was evident with 50/50 hydrogels, most likely due to rFas entrapment within these gels (due to greater pore sizes) (FIG. 4B&C).

The ability of DX2-conjugated hydrogels to induce apoptosis within Jurkat cells, a model T cell line, was assessed by monitoring Annexin V binding. Annexin V binds to phosphatidylserine, which is usually located on the inner leaflet of the phospholipids bilayer of the cell membrane, but becomes translocated to the outer leaflet in dying cells. Binding of Annexin-FITC conjugates to apoptotic Jurkat cells can be visualized by fluorescence microscopy. Counterstaining of cells with propidium iodide can differentiate between apoptotic and necrotic cells, as PI can diffuse into and bind with the DNA only within necrotic cells. In our experiments, relatively few cells (less than 1%) were observed to be necrotic throughout all conditions tested, and therefore only apoptosis results are shown and discussed.

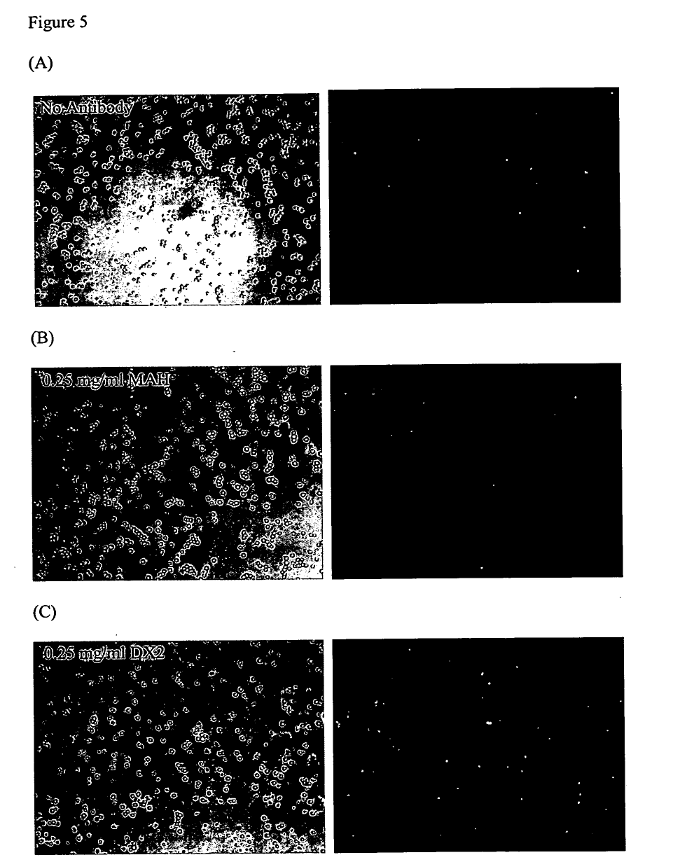

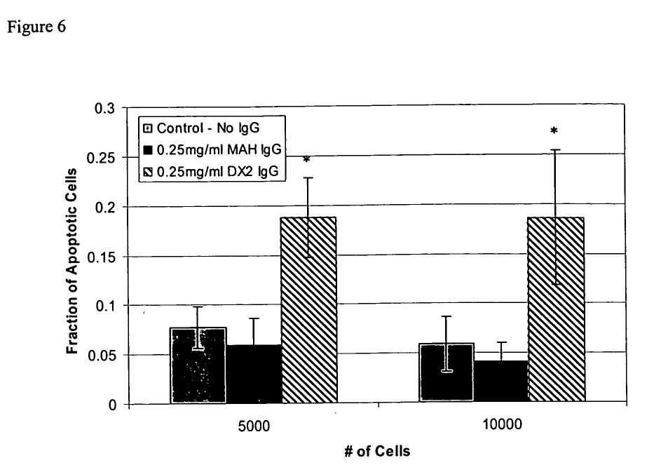

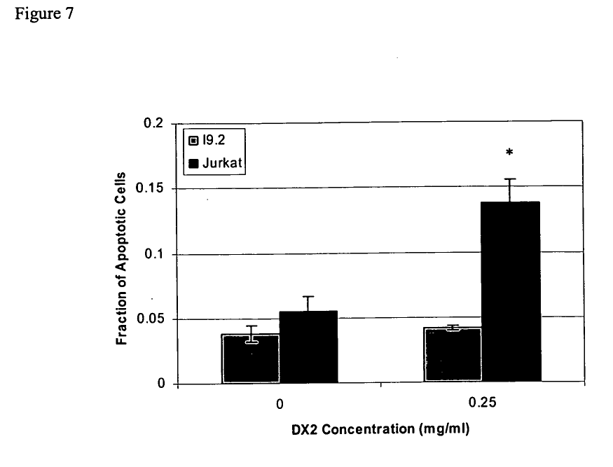

Experiments were performed to compare the ability of DX2-conjugated hydrogels to induce apoptosis within Jurkat cells compared to control hydrogels. Control hydrogels were either unconjugated or were conjugated with MAH IgG control antibodies that are not apoptosis-inducing. Fluorescence images screening for Annexin V fluorescence showed that while similar fractions of cells appear to induce apoptosis in the presence of both control hydrogel formulations, a significant increase in apoptotic cells was observed with hydrogels conjugated with DX2 (Compare fluorescence images from FIG. 5A&B with 5C). These cell images were also used for quantitation of the fraction of apoptotic cells within each condition tested. Quantitative results confirm that an enhancement in apoptosis induction was observed with Jurkat cells exposed to DX2-conjugated gels. Only 5-7.5% of Jurkat cells were apoptotic in the presence of control gels, whereas approximately 20% of Jurkats appeared apoptotic with DX2-conjugated gels (FIG. 6). Statistical analysis by student's t-test showed that all p-values were lower than 0.05, indicating statistical significance of the data. Other tests were performed to test the specificity of apoptosis induction by DX2 antibodies. I9.2 Fas-insensitive T cells were exposed to DX2-conjugated hydrogels to determine whether apoptosis induction occurred due to the specific action of the Fas/anti-Fas interaction or whether apoptosis induction was a non-specific occurrence. Results from this study show that no enhancement in apoptosis was observed with I9.2 cells exposed to 0.25 mg/ml DX2 over levels induces with control gels lacking conjugated antibodies, whereas higher levels of apoptosis were observed within Jurkat cells as expected (FIG. 7).

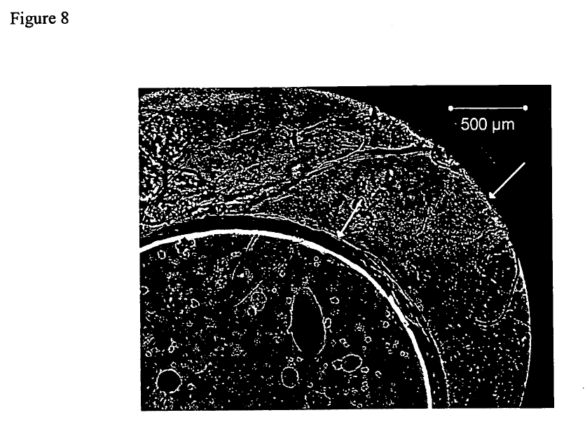

Further studies were performed testing the ability of DX2-conjugated hydrogels containing encapsulated islets to allow for proper islet function while protecting the islets from Jurkat cells exposed to the hydrogels. Balb/c murine pancreatic islets were encapsulated within PEGDA hydrogels in a multilayered fashion, with islets encapsulated within a smaller interior 10% (wt) PEGDA hydrogel surrounded by a larger exterior 10% PEGDA-co-NPA hydrogel conjugated to DX2 MAbs. FIG. 8 represents a brightfield image of this double-layered hydrogel device containing encapsulated islets.

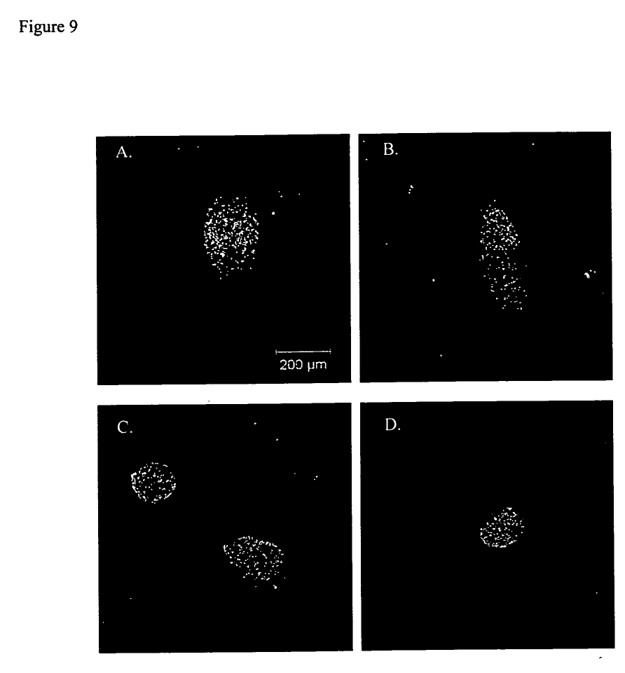

Encapsulated islet viability was assessed by LIVE/DEAD® staining of hydrogels containing islets one day following exposure of the hydrogels to Jurkat T cells. Islets remained viable in both unconjugated and DX2-conjugated hydrogels, as indicated by the intense green calcein staining and minimal red ethidium staining observed within these islets (FIG. 9). These results indicated that the presence of the DX2 MAbs had no deleterious effects on encapsulated islet viability.

Insulin secretion levels from encapsulated islets were similar for both unconjugated and DX-2 conjugated hydrogels (71.2±34.0 ng/ml vs. 96.9±14.6 ng/ml insulin secreted). A student's T-test showed no significant difference between the two insulin secretion levels (p-value=0.47). This indicates that the islets encapsulated in hydrogels containing surface-conjugated DX2 MAbs were as effective in their insulin response to high glucose levels as islets encapsulated in non-conjugated hydrogels.

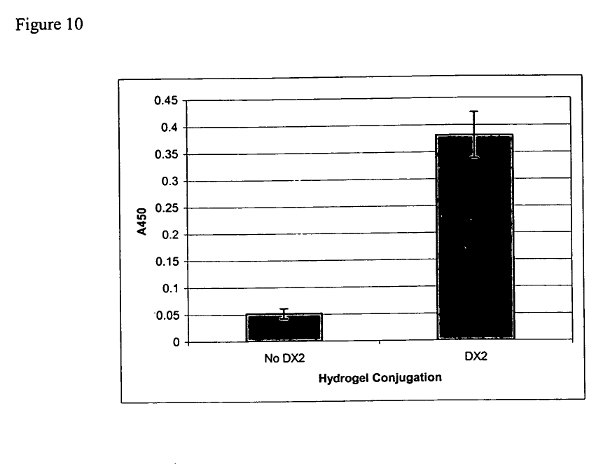

Indirect ELISA results verified that DX2-conjugated islet encapsulated hydrogels indeed contained recognizable DX2 on the surface of these hydrogels, with minimal background absorbance evident with unconjugated hydrogels (FIG. 10). This level of conjugation represented 50% of the DX2 level expected when compared to previous indirect ELISA experiments (see FIG. 4A).

Apoptosis induction within Jurkat T cells following two-day exposure to hydrogels was assessed by Annexin V-FITC conjugate binding. Very few cells were positive for PI binding, indicating that few cells were necrotic in any given sample. Twice as many Jurkat cells stained positive for apoptosis when exposed to DX2-conjugated hydrogels containing encapsulate islets compared to unconjugated hydrogels containing islets (10.2±2.6% apoptotic cells vs. 5.1±1.9% apoptotic cells, respectively), verifying that induction of apoptosis with our model T cells was enhanced in the presence of surface-conjugated DX2 MAbs.

What is claimed is:

1. An immunoisolative encapsulation system comprising a plurality of biological cells encapsulated within a polymer matrix, said polymer matrix covalently bound to a plurality of Fas receptor binding agents.

2. The system of claim 1, wherein said plurality of Fas receptor binding agents is a plurality of anti-Fas antibodies.

3. The system of claim 1, wherein said plurality of Fas receptor binding agents is a plurality of Fas ligand proteins.

4. The system of claim 2, wherein said plurality of anti-Fas antibodies is a plurality of IgG or IgM anti-Fas antibodies.

5. The system of claim 1, wherein said plurality of biological cells is a plurality of secretory cells.

6. The system of claim 1, wherein said plurality of biological cells is a plurality of hepatocytes, parathyroid cells, pancreatic cells, or transformed cell lines.

7. The system of claim 6, wherein said plurality of pancreatic cells is a plurality of islet cells.

8. The system of claim 1, wherein said polymer matrix is a hydrogel.

9. The system of claim 8, wherein said hydrogel comprises polyethylene glycol polymers.

10. The system of claim 8, wherein said hydrogel is a PEGDA-co-NPA hydrogel.

11. The system of claim 1, wherein the plurality of Fas receptor binding agents are covalently bound to the surface of the polymer matrix.

12. A method of inducing apoptosis in a T-cell, said method comprising contacting said T-cell with the immunoisolative encapsulation system of claim 1, and allowing at least one of said plurality of Fas receptor binding agents to bind to a Fas receptor on the surface of said T-cell.

13. The method of claim 12, wherein said contacting is performed in vivo.

14. The method of claim 12, wherein said contacting is performed in a mammal.

15. The method of claim 12, wherein said contacting is performed in a human.

16. A method of treating Type I diabetes mellitus in a subject in need of such treatment, said method comprising
(1) transplanting an immunoisolative encapsulation system into said subject, wherein the immunoisolative encapsulation system comprises a plurality of islet cells encapsulated within a polymer matrix covalently bound to a plurality of Fas receptor binding agents, and
(2) allowing the beta cells of said plurality of islet cells to secrete insulin thereby treating said subject.

17. A method of locally suppressing an immune response to a transplanted biological material in a subject, said method comprising the steps of:
(a) encapsulating said transplanted biological material within a polymer matrix covalently bound to a plurality of Fas receptor binding agents thereby forming an immunoisolative encapsulation system;
(b) transplanting said immunoisolative encapsulation system into said subject;
(c) allowing at least one of said plurality of Fas receptor binding agents to bind to one or more Fas receptors on the surface of a plurality of T-cells, thereby locally suppressing the immune response to the transplanted biological material.

18. The method of claim 17, wherein the transplanted biological material is transplanted hepatocytes, parathyroid cells, or pancreatic cells.

19. The method of claim 17, wherein the transplanted biological material is transplanted islet cells.
